(19) United States
(12) Patent Application Publication
(10) Pub. No.: US 2009/0245458 A1
(43) Pub. Date: Oct. 1, 2009

(54) EXTENSION OF THE Q-RANGE IN CSCT

(75) Inventors: Udo Van Stevendaal, Ahrensburg (DE); Jens-Peter Schlomka, Hamburg (DE); Axel Thran, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/300,640

(22) PCT Filed: May 8, 2007
(86) PCT No.: PCT/IB07/51718

§ 371 Date: Nov. 13, 2008

(30) Foreign Application Priority Data
May 16, 2006 (EP) 06114051.3

(51) Int. Cl. A61B 6/00; G06T 17/00

(52) U.S. Cl. 378/14; 345/424

(57) ABSTRACT

According to an embodiment of the present invention, a reconstruction scheme for a CSCT is provided which is capable of reconstructing data acquired just over a half turn, i.e. 180° plus fan angle. The reconstruction scheme may provide for an extension of the reconstruction towards smaller and larger q-regions as compared with a 360°-reconstruction, if the short scan reconstruction technique is applied to a full scan data set.

The invention relates to the field of tomographic imaging. In particular, the invention relates to a coherent scatter computed tomography apparatus for examination of an object of interest, a method of examination of an object of interest, an image processing device, a computer-readable medium and a program element.

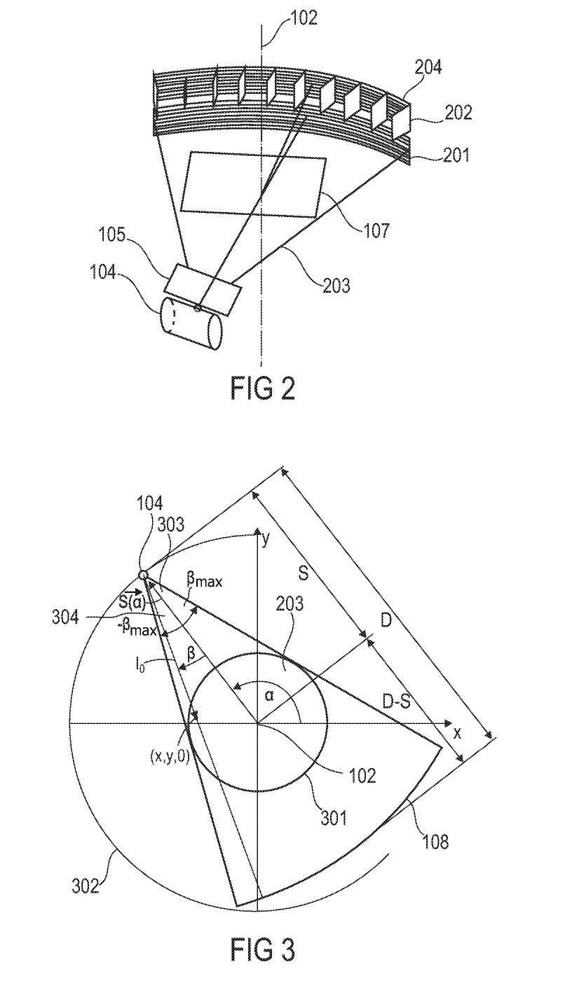

In coherent scatter computed tomography (CSCT), a narrow fan-beam with small divergence in the out-of-fan plane direction or a focused fan-beam penetrates an object of interest. The transmitted radiation as well as the radiation scattered in the direction out-of-the fan plane is detected. FIG. 2 shows the geometry of an exemplary set-up with a separate energy-resolving scatter detector.

The combined CT and scatter information may be used for material identification in the case of baggage inspection applications and in medical applications for the detection of diseases, which modify the molecular structure of tissue.

However, current CSCT reconstruction algorithms require a data acquisition over 360°. In this case, the completeness condition is fulfilled and the data need no further weighting since all voxels are exposed uniformly. Performing the acquisition over a full turn may not only assure the completeness condition but may also yield the correct weighting of the data. However, the 360° completeness condition is responsible for a q-range limitation to small as well as to large values due to the scanner geometries.

It would be desirable to have an improved reconstruction scheme for CSCT.

The invention provides a coherent scatter computed tomography apparatus, an image processing device, a computer-readable medium, a program element and a method of examining an object of interest with the features according to the independent claims.

It should be noted that the following described exemplary embodiments of the invention apply also for the method of examination of the object of interest, for the computer-readable medium, for the image processing device and for the program element.

According to a first aspect of the present invention, a coherent scatter computed tomography apparatus for examination of an object of interest is provided, the coherent scatter computed tomography apparatus comprising a reconstruction unit adapted for selecting, from a data set, a voxel and a q-range, weighting a projection comprising the voxel and corresponding to a q-value within the q-range, resulting in a weighted projection, and back-projecting the weighted projection.

Advantageously, the coherent scatter computed tomography apparatus may be adapted for reconstructing data acquired just over a half turn, i.e. 180° plus fan angle. Furthermore, the CSCT apparatus may be adapted for extending the reconstruction towards smaller and larger q-regions, respectively, compared to a 360° reconstruction.

The CSCT apparatus may be further adapted for performing a divergent convolution after the weighting of the projection, wherein the back-projecting is performed along curved lines.

Therefore, a divergent convolution and back-projection along curved lines are performed for image reconstruction after the weighting.

In an embodiment of this aspect of the present invention, the weighting is a Parker weighting.

Hence, a smooth weighting of data in double-scanned regions is performed, wherein the weights and their derivatives are continuous at the boundaries.

According to another embodiment of the present invention, the selected voxel and the q-range do not fulfil a 360° condition but fulfil at least a 180° condition.

According to another embodiment of the present invention, the CSCT apparatus is further adapted for performing a coherent scatter computed tomography data acquisition over a 360° rotation of the radiation source, resulting in the data set. The CSCT apparatus is further adapted for evaluating an angular range which has been seen by any individual voxel and q-value and for performing a 360°-reconstruction for all voxels and q-values which fulfil a 360° completeness condition.

Therefore, according to this embodiment of the present invention, the above 180° reconstruction algorithm may be used to extend the accessible q-range in all spatial directions when applied to a full 360° data set.

According to another embodiment of the present invention, the CSCT apparatus is configured as one of the group consisting of a material testing apparatus and a medical application apparatus. A field of application of the invention may be medical imaging or baggage inspection.

According to another embodiment of the present invention, the CSCT apparatus further comprises a collimator arranged between the electromagnetic radiation source and the detector unit, wherein the collimator is adapted for collimating an electromagnetic radiation beam emitted by the electromagnetic radiation source to form a cone-beam or a fan-beam.

Furthermore, the CSCT apparatus may be adapted as an energy-resolved coherent scatter computed tomography apparatus.

Furthermore, according to another embodiment of the present invention, a method of examination of an object of interest with a coherent scatter computed tomography apparatus is provided, the method comprising the steps of selecting, from a data set, a voxel and a q-range, weighting a projection comprising the voxel and corresponding to a q-value within the q-range, resulting in a weighted projection, and back-projecting the weighted projection.

This may provide for a reduction of measurement time if just a half scan is performed. Furthermore, performing a full scan combined with the half scan reconstruction method may lead to an extension of the q-range.

According to another embodiment of the present invention, an image processing device for examination of an object of interest may be provided, the image processing device comprising a memory for storing a data set of the object of interest and a reconstruction unit adapted for carrying out the above-mentioned method steps.

According to another embodiment of the present invention, a computer-readable medium may be provided, in which a computer program for examination of an object of interest is stored which, when executed by a processor, causes said processor to carry out the above-mentioned method steps.

Furthermore, according to another embodiment of the present invention, a program element for examination of an object of interest may be provided, which, when executed by a processor, causes said processor to carry out the above-mentioned method steps.

Those skilled in the art will readily appreciate that the method of examination of the object of interest may be embodied as the computer program, i.e. by software, or may be embodied using one or more special electronic optimization circuits, i.e. in hardware, or the method may be embodied in hybrid form, i.e. by means of software components and hardware components.

The program element according to an embodiment of the invention is preferably loaded into working memories of a data processor. The data processor may thus be equipped to carry out embodiments of the methods of the present invention. The computer program may be written in any suitable programming language, such as, for example, C++ and may be stored on a computer-readable medium, such as a CD-ROM. Also, the computer program may be available from a network, such as the Worldwide Web, from which it may be downloaded into image processing units or processors, or any suitable computers.

It may be seen as the gist of an exemplary embodiment of the present invention that a CSCT reconstruction is performed on the basis of a data set acquired just over a half turn of the gantry, i.e. 180° plus fan angle. A feature of the new reconstruction scheme is that it may provide an extension of the reconstruction towards smaller and larger q-regions, respectively, compared to a 360° reconstruction.

These and other aspects of the present invention will become apparent from and elucidated with reference to the embodiments described hereinafter.

The invention will now be described, by way of example only, with reference to the accompanying drawings, in which:

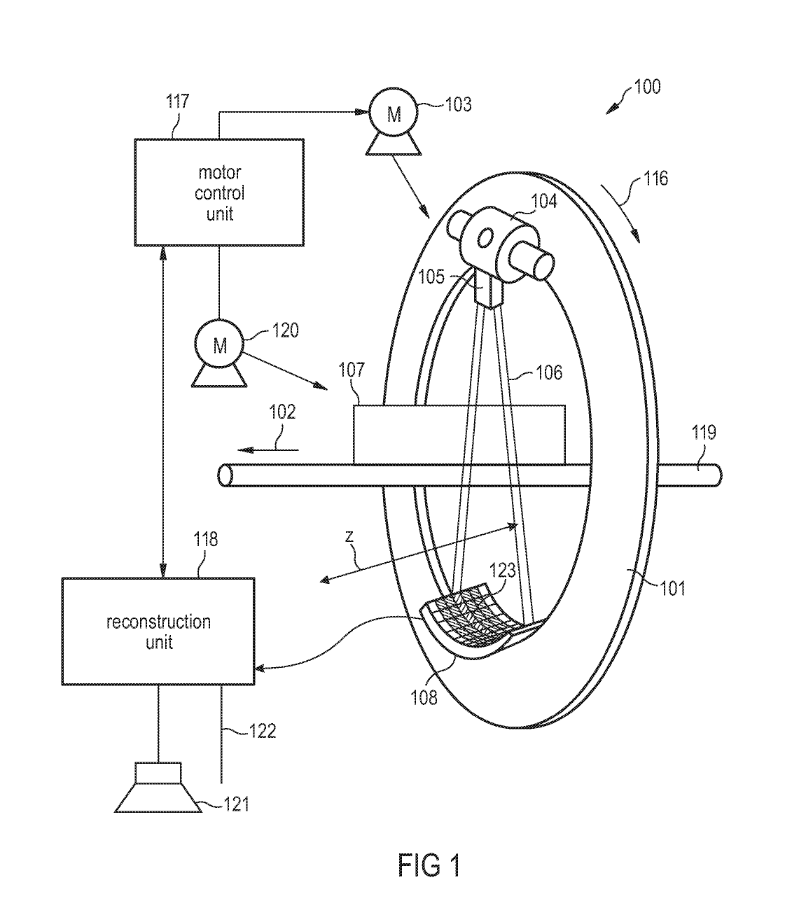

FIG. 1 shows a simplified schematic representation of a CSCT apparatus according to an exemplary embodiment of the present invention.

FIG. 2 shows a schematic representation of a geometry for CSCT.

FIG. 3 shows a schematic representation of a CSCT geometry in the x-y plane.

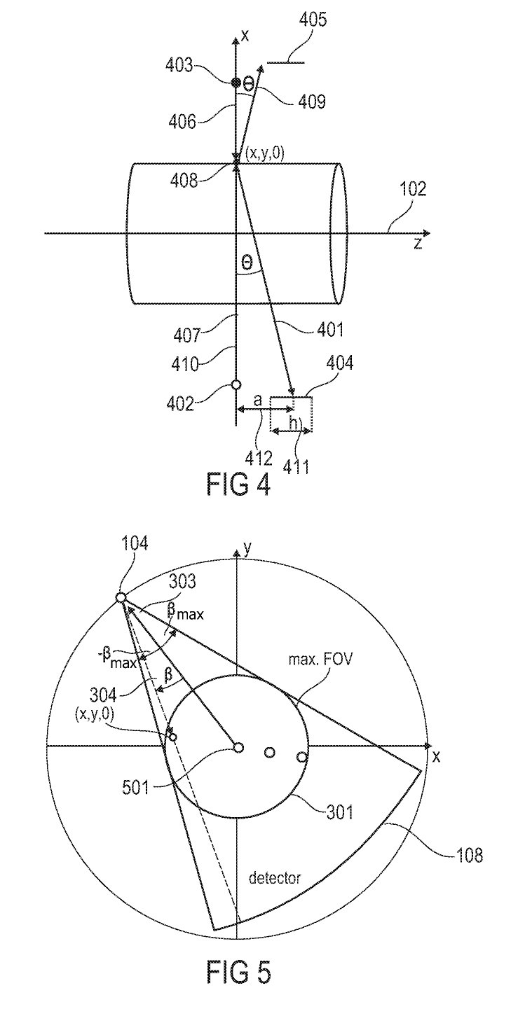

FIG. 4 shows a schematic representation of a CSCT geometry in the x-z plane.

FIG. 5 shows a schematic representation of a CSCT geometry in the x-y plane.

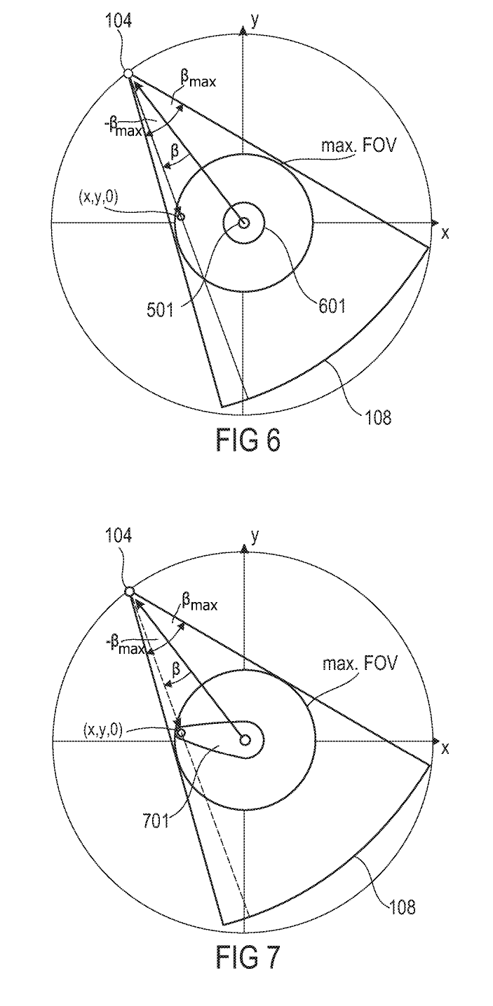

FIG. 6 shows a schematic representation of a CSCT geometry in the x-y plane.

FIG. 7 shows a schematic representation of a CSCT geometry in the x-y plane.

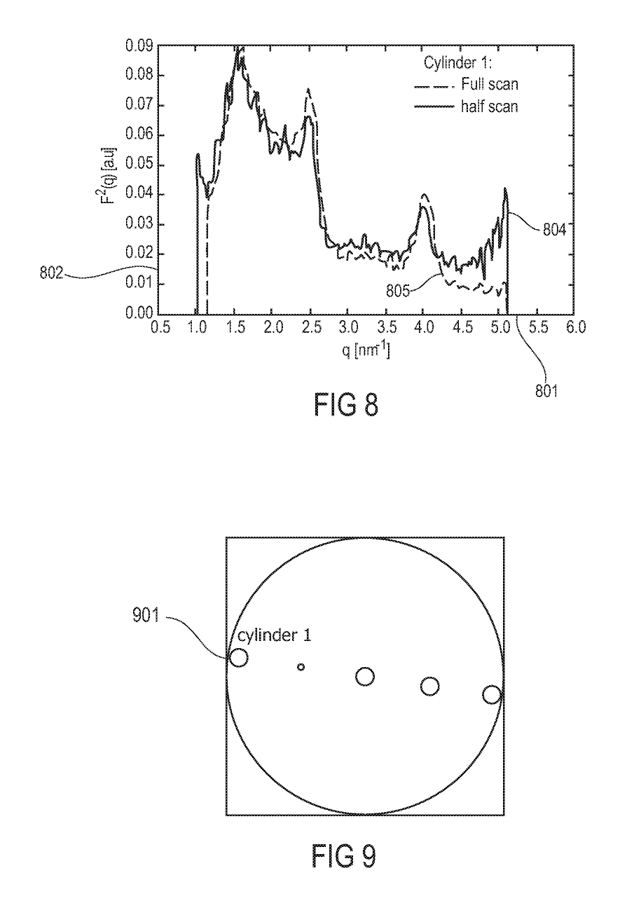

FIG. 8 shows a scatter function F² (q) of the cylinder depicted in FIG. 9.

FIG. 9 shows a schematic representation of the cylinder corresponding to the data depicted in FIG. 8.

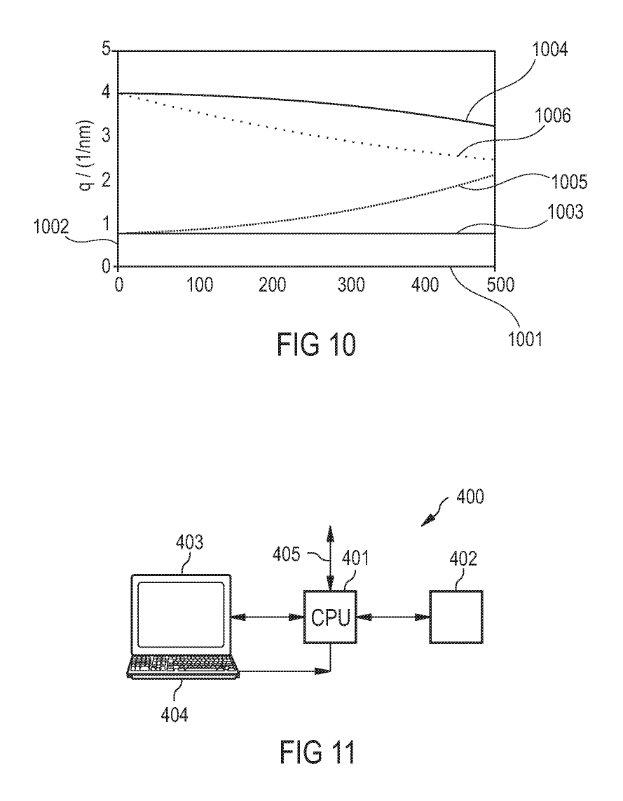

FIG. 10 shows lower and upper limits of the momentum transfer ranges which can be reconstructed.

FIG. 11 shows an exemplary embodiment of an image processing device according to the present invention, for executing an exemplary embodiment of a method in accordance with the present invention.

In different drawings, similar or identical elements are provided with the same reference numerals.

FIG. 1 shows an exemplary embodiment of a CT/CSCT scanner system according to an exemplary embodiment of the present invention. With reference to this exemplary embodiment, the present invention will be described for the application in the field of baggage inspection. However, it should be noted that the present invention is not limited to this application, but may also be applied in the field of medical imaging, or other industrial applications, such as material testing.

The computer tomography apparatus 100 depicted in FIG. 1 is a fan-beam CT/CSCT scanner. The CT/CSCT scanner depicted in FIG. 1 comprises a gantry 101, which is rotatable around a rotational axis 102. The gantry 101 is driven by means of a motor 103. Reference numeral 104 designates a source of radiation such as an X-ray source, which, according to an aspect of the present invention, emits a polychromatic radiation.

Reference numeral 105 designates an aperture system which forms the radiation beam emitted from the radiation source to a fan-shaped radiation beam 106. The fan-beam 106 is directed such that it penetrates an object of interest 107 arranged in the centre of the gantry 101, i.e. in an examination region of the CT/CSCT scanner, and impinges onto the detector 108. As may be taken from FIG. 1, the detector 108 is arranged on the gantry 101 opposite to the source of radiation 104, such that the surface of the detector 108 is at least partially illuminated by the fan-beam 106. The detector 108, which is depicted in FIG. 1, comprises a plurality of detector elements 123 each capable of detecting, in an energy-resolving or non-energy resolving manner, X-rays or individual photons which have penetrated the object of interest 107.

During a scan of the object of interest 107, the source of radiation 104, the aperture system 105 and the detector 108 are rotated along the gantry 101 in the direction indicated by arrow 116. For rotation of the gantry 101 with the source of radiation 104, the aperture system 105 and the detector 108, the motor 103 is connected to a motor control unit 117, which is connected to a calculation or reconstruction unit 118.

In FIG. 1, the object of interest 107 may be an item of baggage or a patient which is disposed on a conveyor belt 119. During the scan of the object of interest 107, while the gantry 101 rotates around the item of baggage 107, the conveyor belt 119 is stopped. By this, the object of interest 107 is scanned along a circular scan path. Instead of providing a conveyor belt 119, for example, in medical applications where the object of interest 107 is a patient, a movable table may be used. However, it should be noted that in all of the described cases it may also be possible to perform other scan paths.

The detector 108 may be connected to the calculation unit 118. The calculation unit 118 may receive the detection result, i.e. the read-outs from the detector elements 123 of the detector 108 and may determine a scanning result on the basis of the read-outs. Furthermore, the calculation unit 118 communicates with the motor control unit 117 in order to coordinate the movement of the gantry 101 with motors 103 and 120 with the conveyor belt 119.

The calculation unit 118 may be adapted for performing an image reconstruction, according to an exemplary embodiment of the present invention. A reconstructed image generated by the calculation unit 118 may be output to a display (not shown in FIG. 1) via an interface 122.

The calculation unit 118 may be realized by a data processor to process read-outs from the detector elements 123 of the detector 108.

Furthermore, as may be taken from FIG. 1, the calculation unit 118 may be connected to a loudspeaker 121, for example, to automatically output an alarm in case of the detection of suspicious material in the item of baggage 107.

The computer tomography apparatus 100 for examination of the object of interest 107 includes the detector 108 having the plurality of detecting elements 123 arranged in a matrix-like manner, each being adapted to detect X-rays. Furthermore, the computer tomography apparatus 100 comprises the determination unit or reconstruction unit 118 adapted for reconstructing an image of the object of interest 107.

The computer tomography apparatus 100 comprises the X-ray source 104 adapted to emit X-rays to the object of interest 107. The collimator 105 provided between the electromagnetic radiation source 104 and the detecting elements 123 is adapted to collimate an electromagnetic radiation beam emitted from the electromagnetic radiation source 104 to form a fan-beam. The detecting elements 123 form a multi-slice detector array 108.

FIG. 2 shows a schematic representation of a geometry for CSCT. A central detector 201, which is adapted as a single-line or as a multi-line detector, detects the radiation which is directly transmitted from the x-ray tube 104 and collimated by a fan-beam collimator 105, thereby forming a fan-beam 203. Furthermore, one-dimensional scatter collimators 202 and a detector 204 are provided offset the fan-beam 203. The CSCT-detector 204 can be adapted as an energy-resolving or non-energy resolving detector and measures scattered radiation.

It is known from CT algorithms that data from half turn, i.e. 180° plus fan angle, are sufficient enough for an appropriate image reconstruction. Disadvantageously, during rotation around the object of interest 107, a distance between the object 107 and the detector 201, 204 changes and thus the scatter angle between a fixed object voxel and a given detector row changes as well.

When a multi-line scatter detector is used, a range of scatter angles is measured simultaneously. From a scatter angle Θ and a photon energy E the momentum transfer parameter q is calculated,

$$q = \frac{E}{hc} sin ( Θ / 2 ) \qquad ( equation 1 )$$

wherein h is Planck's constant and c the speed of light.

The accessible energy range is limited by a minimum energy Eₘᵢₙ and a maximum energy Eₘₐₓ, e.g. Eₘᵢₙ=50 keV . . . Eₘₐₓ=100 keV. Thus, for each scatter angle Θ only a finite q-range is measured. Reconstruction may only be performed for that range of q-values, which reach the detector for all rotational steps.

According to an aspect of the present invention, the number of required rotational steps is reduced by up to 50%. As a consequence, the range of scatter angles allowing reconstruction is extended and thus the accessible q-range. A larger accessible q-range allows an improved detection of materials in baggage inspection applications or a better discrimination between healthy and disease tissue in medical applications.

FIG. 3 shows a schematic representation of a CSCT geometry in the x-y plane. The grey filled circle 301 indicates the field of view (FOV). The segment 302 of the big circle symbolizes the trajectory for a half scan, i.e. 180° plus fan angle.

The field of view 301 in the x-y plane is given by the scanner geometry and by the width of the detector 108 in particular. The accessible q-range is limited by the field of view and also depends on the scanner geometry, the detector height, and the measurable energy range, as depicted in FIG. 4. With smaller or larger q-values, the field of view decreases because the voxel at the edge of the field of view do not fulfil the 360°-condition any longer.

FIG. 4 shows a schematic representation of a CSCT geometry in the x-z plane. The detector height h and the distance a of the detector from the central plane 406 are crucial for the q-range.

Reference numeral 402 shows the source position at 180°, reference numeral 403 shows the source position at 0°. 407 depicts the path of an incoming ray emitted from the source 402 at 180°. 406 shows the path of an incoming ray emitted from the source 403 at 0°. The incoming ray 406 is scattered at object point 408 in direction 401 towards the detector, which is positioned at 0° 404. On the other hand, if the source is positioned at 180° 402, the incoming ray 407 is scattered by object point 408 in direction 409 and does not hit the detector which is positioned at 180° 405.

A reduction of the acquisition angle to 180° plus fan angle may be achieved by introducing a reconstruction algorithm with an appropriate weighting of the detector data. Furthermore, this reconstruction algorithm may be used for the extension of the q-range in both directions, i.e. for smaller as well as for larger q-values when applied to data from a 360° scan.

In the following, a reconstruction algorithm for half scan acquisition and a method of extension of the q-range, according to exemplary embodiments of the present invention, are described in more detail:

Reconstruction algorithm for half scan acquisition

It is known from CT reconstruction algorithms that data obtained from a set of divergent ray projections taken over 180° plus the angle of the divergent fan-beam form the minimal complete data set. It is the minimum set of equally spaced projection measurements, which can be used in conventional convolution type reconstruction algorithms. According to an aspect of the present invention, the CSCT 360°-reconstruction scheme is extended by a 180°-reconstruction algorithm by introducing a Parker weighting,

$$ω ( α , β ) = {sin}^{2} ( \frac{π}{4} \frac{α}{{β}_{ma x} - β} ) , \\ 0 \leq α \leq 2 {β}_{ma x} - 2 β \\ ω ( α , β ) = 1 , \\ 2 {β}_{ma x} - 2 β \leq α \leq π - 2 β \\ ω ( α , β ) = {sin}^{2} ( \frac{π}{4} \frac{π + 2 {β}_{ma x} - α}{{β}_{ma x} + β} ) , \\ π - 2 β \leq α \leq π + 2 {β}_{ma x} , \qquad ( equation 2 )$$

where a denotes the rotational angle of the source, βₘₐₓ denotes the fan angle 303, and β denotes the angle of the ray within the fan which exposes the voxel of interest, as depicted in FIG. 3 by reference numeral 304.

Parker weighting is described in “Optimal short scan convolution reconstruction for fan beam CT”, D. L. Parker, Med. Phys. 9(2), March/April 1982, pp. 254-257, which is hereby incorporated by reference.

As can be seen, the technique may require the smooth weighting of data in double-scanned regions while the weights and their derivatives are required to be continuous at the boundaries. After the weighting is performed, divergent convolution and back-projection along curved lines are performed to reconstruct the image.

Extension of the q-range

As already mentioned, the limitation of the q-range is given by the distance of the detector to the central plane 410, by the detector height 411 and by the energy range of the detector. Without a loss of generality, we consider just one detector row with the distance a 412 to the central plane according to FIG. 4. If we assume that the object point at (x,y) scatters photons under an angle Θ onto the detector at α=0°, the completeness condition requires that the detector receives radiation of the scatter centre from each source position during a full turn. However, this is not necessarily the case if the source-detector unit has reached the opposite position, 180° for example. The radiation reaching the detector has now been scattered under a different angle. To measure the same q-value according to equation 1, a different energy has to be used. However, this new energy may not be contained in the range Eₘᵢₙ . . . Eₘₐₓ. Thus, this q-value cannot be reconstructed in a 360° case, as also illustrated in FIGS. 5 and 6.

FIG. 5 shows a CSCT geometry in the x-y plane. The central region 501 shows the voxel region which is exposed over 360°. 301 shows the maximum field of view. While the maximal field of view can be imaged for a wave-vector transfer q=2.5 nm⁻¹, as depicted in FIG. 5, voxel with a larger distance from the centre of rotation do not fulfil the completeness condition for q=1.04 nm⁻¹ (as depicted in FIG. 6). In this case, just a small rotationally symmetrical region 601 can be reconstructed, wherein in the case of FIG. 5, the maximum field of view 301 can be reconstructed.

If we further assume that the scattered photons from the source-detector positions over the angle regime from 90° to 270° plus fan angle reach the detector, this object point can be reconstructed with the half scan reconstruction method.

FIG. 7 shows a schematic representation of a CSCT geometry in the x-y plane. In FIG. 7, it is demonstrated that an additional part can be taken into account for the reconstruction process. Voxel from the outer region fulfil the condition necessary for a 180°-reconstruction. This can be done for several segments of the field of view which leads to an extension of the q-range as shown in FIG. 8. In other words, as may be seen from FIG. 7, at the wave-vector transfer q=1.04 nm⁻¹, a larger region of the maximum field of view can be reconstructed with the half scan reconstruction method compared to the full scan reconstruction method.

Reference numeral 701 indicates the voxel region which is exposed over 180° plus fan angle.

FIG. 8 shows a scatter function F²(q) of the cylinder 901 depicted in FIG. 9 reconstructed with the conventional 360° reconstruction method 804 (full scan) and with the 180° reconstruction method 805. Horizontal axis 801 represents the q-value, ranging from 0.5 to 6.0 nm⁻¹ Vertical axis 802 shows the scatter function in arbitrary units.

While the scatter function F²(q) of cylinder 901 can only be reconstructed down to q=1.18 nm⁻¹ with a 360°-reconstruction method it is extended to q=1.04 nm⁻¹ with the help of the described 180°-reconstruction method. The effect is bigger the larger the given field of view is.

FIG. 9 shows a schematic representation of the cylinder 901 corresponding to the scatter function depicted in FIG. 8.

FIG. 10 shows the lower and upper limits of the momentum transfer ranges which can be reconstructed in case of the 360°-reconstruction (1005, 1006, respectively) and with respect to the proposed 180°-reconstruction (1003, 1004, respectively). A scatter detector covering a height range from 32 to 80 mm measured from the fan plane at an energy range of evaluable photons from 50 to 100 keV has been assumed for the calculations.

The horizontal axis 1001 shows the radius of the field of view in mm in ranges from 0 to 500. The vertical axis 1002 shows the q-values ranging from 0 to 5 nm⁻¹. In the 360° case the momentum transfer range shrinks considerably, when the field of view increases, while in the 180° case the lower limit of the q-range is independent of the size of the field of view and the dependence of the upper limit on the field of view is much smaller than in the 360° case.

The new 180° reconstruction scheme may be used to extend the accessible q-range in all spatial directions when applied to a full 360° data set by the following method:

Firstly, a CSCT measurement is performed with a data acquisition over a 360° rotation. Then, in step 2, the angular range, which has been seen by any individual voxel and q-value, is evaluated. In step 3, a conventional 360°-reconstruction is performed for all voxels and q-values, which fulfil the 360° completeness condition. Finally, in step 4, all voxel and q-range are chosen, which do not fulfil the 360° condition but which fulfil at least the 180° condition and an appropriate weighting is applied to all projections containing this voxel/q-value, followed by a back-projection.

FIG. 11 depicts an embodiment of a data processing device 400 for executing a method aspect of the present invention. The data processing device 400 depicted in FIG. 11 comprises a central processing unit (CPU) or image processor 401 connected to a memory 402 for storing an image depicting an object of interest, such as a patient or an item of baggage. The data processor 401 may be connected to a plurality of input/output network or diagnosis devices, such as a CT device. The data processor 401 may furthermore be connected to a display device 403, for example, a computer monitor, for displaying information or an image computed or adapted in the data processor 401. An operator or user may interact with the data processor 401 via a keyboard 404 and/or other output devices, which are not depicted in FIG. 11.

Furthermore, via the bus system 405, it may also be possible to connect the image processing and control processor 401 to, for example, a motion monitor, which monitors a motion of the object of interest. In case, for example, a lung of a patient is imaged, the motion sensor may be an exhalation sensor. In case the heart is imaged, the motion sensor may be an electrocardiogram.

The above-described aspects may be used for energy-resolved or non-energy-resolved CSCT, and for fan-beam or cone-beam CSCT. Furthermore, the applications of these methods may be in, but are not limited to, the medical field as an add-on for CT and in baggage inspection applications for unambiguous and fast material identification.

It should be noted that the term “comprising” does not exclude other elements or steps and the “a” or “an” does not exclude a plurality. Also elements described in association with different embodiments may be combined.

It should also be noted that reference signs in the claims shall not be construed as limiting the scope of the claims.

1. A coherent scatter computed tomography apparatus for examination of an object of interest (107), the coherent scatter computed tomography apparatus comprising:
a reconstruction unit (118) adapted for:
selecting, from a data set, a voxel and a q-range, such that the selected voxel and the q-range do not fulfil a 360° condition but fulfil at least a 180° condition;
weighting a projection comprising the voxel and corresponding to a q-value within the q-range, resulting in a weighted projection;
back-projecting the weighted projection.

2. The coherent scatter computed tomography apparatus of claim 1, further adapted for:
performing a divergent convolution after the weighting of the projection;
wherein the back-projecting is performed along curved lines.

3. The coherent scatter computed tomography apparatus of claim 1, wherein the weighting is a Parker weighting.

4. (canceled)

5. The coherent scatter computed tomography apparatus of claim 1, further comprising:
a radiation source (104);
wherein the coherent scatter computed tomography apparatus is further adapted for:
performing a coherent scatter computed tomography data acquisition over a 360° rotation of the radiation source, resulting in the data set;
evaluating an angular range which has been seen by any individual voxel and q-value;
performing a 360°-reconstruction for all voxels and q-values which fulfil a 360° completeness condition;
performing a 180°-reconstruction for all voxels and q-values which do not fulfil a 360° completeness condition but fulfil the 180° completeness condition.

6. The coherent scatter computed tomography apparatus of claim 1, configured as one of the group consisting of a material testing apparatus, a medical application apparatus.

7. The coherent scatter computed tomography apparatus of claim 1, further comprising:
detector unit (108); and
a collimator (105) arranged between the electromagnetic radiation source (104) and the detector unit (108);
wherein the collimator (105) is adapted for collimating an electromagnetic radiation beam emitted by the electromagnetic radiation source (104) to form a cone-beam or a fan-beam.

8. The coherent scatter computed tomography apparatus of claim 1, adapted as an energy-resolved coherent scatter computed tomography apparatus.

9. Method of reconstructing an image of an object of interest on the basis of a coherent scatter computed tomography data set, the method comprising the steps of:
selecting, from the data set, a voxel and a q-range, such that the selected voxel and the q-range do not fulfil a 360° condition but fulfil at least a 180° condition;
weighting a projection comprising the voxel and corresponding to a q-value within the q-range, resulting in a weighted projection;
back-projecting the weighted projection.

10. Method of claim 9, further comprising the steps of:
evaluating an angular range which has been seen by any individual voxel and q-value;
performing a 360°-reconstruction for all voxels and q-values which fulfil a 360° completeness condition;
performing a 180°-reconstruction for all voxels and q-values which do not fulfil a 360° completeness condition but fulfil the 180° completeness condition.

11. An image processing device for examination of an object of interest, the image processing device comprising:
a memory for storing a data set of the object of interest (107);
a reconstruction unit (118) adapted for:
selecting, from the data set, a voxel and a q-range such that the selected voxel and the q-range do not fulfil a 360° condition but fulfil at least a 180° condition;
weighting a projection comprising the voxel and corresponding to a q-value within the q-range, resulting in a weighted projection;
back-projecting the weighted projection.

12. A computer-readable medium (402), in which a computer program for examination of an object of interest (107) is stored which, when executed by a processor (401), causes said processor to carry out the steps of:
selecting, from the data set, a voxel and a q-range, such that the selected voxel and the q-range do not fulfil a 360° condition but fulfil at least a 180° condition;
weighting a projection comprising the voxel and corresponding to a q-value within the q-range, resulting in a weighted projection; and
back-projecting the weighted projection.

13. (canceled)
